(19) United States
(12) Patent Application Publication
(10) Pub. No.: US 2005/0232985 A1
(43) Pub. Date: Oct. 20, 2005

(54) Ciprofloxacin hcl

(76) Inventor: Robert Janitzek, Olongapo City (PH)

Correspondence Address: ROTH & GOLDMAN, P.A., 523 W. 6TH STREET, SUITE 707, LOS ANGELES, CA, 90014, US

(21) Appl. No.: 10/519,467

(22) PCT Filed:
(86) PCT No.: PCT/PH03/00006

(30) Foreign Application Priority Data
Jun. 27, 2002 (PH) 1-2002-000485

(51) Int. Cl. A61K 31/496; A61K 9/20

(52) U.S. Cl. 424/464; 514/253.08

(57) ABSTRACT

This invention relates to Ciprofloxacin Hydrochloride-containing composition, useful for the treatment of diseases. In particular, it relates to pharmaceutical composition comprising the following essential components: i) Ciprofloxacin Hydrochloride; ii) Magnesium stearate; iii) Starch; and iv) Carboxymethylstach Sodium.

This invention relates to Ciprofloxacin Hydrochloride-containing compositions, useful for the treatment of diseases.

The main composition is Ciprofloxacin Hydrochloride and the chemical name in accordance with the Merck Index II^{th} Edition, page 360 (1989) has the International Non-propriety Name (INN) for I-cyclopropyl-6-fluoro-1,4-dihydro-4-oxo-7-(I-piperazinyl)-3-quinolinecarboxylic acid of the structural formula: 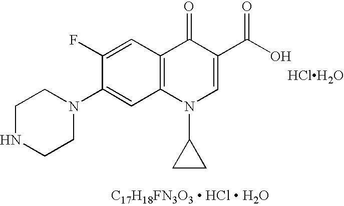
disclosed, together with the process for the preparation thereof, in U.S. Pat. No. 4,670,444. Ciprofloxacin is a broad-spectrum antibacterial active principle.

The Ciprofloxacin Hydrochloride may easily be obtained on the market place or may be prepared by any of the methods disclosed in Spanish Patents Es-2006099 and ES-2006098.

Ciprofloxacin Hydrochloride is a white or off-white powder, which is odorless and has a bitter taste with a wide range antibacterial activity against Excherichia Coli, Klebsiella SPP., and other Enterobacter SPP., Bacillus—negative. The antibacterial action against Pseudomonas aerugenosa, golden yellow Staphylococcus and Streptococcus pneumoniae is better than other known derivatives i.e. Norfloxacin and Peifloxacin but the antibacterial action against Streptococcus SPP. Is less than Penicillin kinds of antibiotic.

The antibacterial action results from the inhibition of bacterial DNA gyrase for combating various types of disorders. Ciprofloxacin Hydrochloride may be used in combination with an amino glycoside or with beta-lactam antibiotics.

The present inventor is aware of the existence of prior art describing ciprofloxacin pharmaceutical preparations which may be used for combating diseases whereby, in view of the high efficacy and broad spectrum of this antibacterial active principle, there is a felt the desirability of developing new compositions containing it, suitable for such application.

The invention seeks to provide aqueous ciprofloxacin compositions suitable for use in the treatment of the following infections caused by sensitive bacteria:
1. Upper respiratory tract infections including tonsillitis, sinusitis, otitis media and pharynx inflammation.
2. Lower respiratory tract infections including acute and chronic bronchitis, bronchiectasis and pneumonia.
3. Urinary tract infections including urethritis, cystitis, pyelonephritis, prostatitis and pelvic inflammatory.
4. Gastro-intestinal infections including enteric fever and infective diarrhea.
5. Skin and soft tissue infections.
6. Wounds infections.
7. Infections caused by other sensitive bacteria.

This objective is achieved by composition according to the present invention characterized in that they comprise the following essential components, in the amounts given hereinafter.
i. 300 g of Ciprofloxacin;
ii. 65 g of starch;
iii. 18 g of Carboxymethyl starch Sodium;
iv. 4 g of Magnesium Stearate

The technical specifications of the component of the present invention are as follows:

2.1 Technical specifications of Ciprofloxacin Hydrochloride

| | Items | Specifications |
|---|---|---|
| 1. | Identification | |
| A. | IR Test | Conform |
| B. | TLC Test | Conform |
| C. | Chloride Test | Conform |
| 2. | pH | 3.0˜4.5 |
| 3. | Water | 4.7˜6.7% |
| 4. | Residue of ignition | □ 0.1% |
| 5. | Sulfate | □ 0.04% |
| 6. | Heavy metals | □ 0.002% |
| 7. | Limit of fluoroquin-olonic acid | □ 0.2% |
| 8. | Chromatographic purity | |
| | Single impurity: | □ 0.2% |
| | Total impurities: | □ 0.5% |
| 9. | Assay | 98.0˜102.0% |

2.2 Technical specifications of Magnesium Stearate

| | Items | Specifications |
|---|---|---|
| 1. | Identification | |
| A. | Magnesium test | Conform |
| B. | The retention time test | Conform |
| 2. | Microbial limits | |
| A. | The total aerobic and microbial count | □ 1000 per g |
| B. | The total combined molds and | □ 500 per g |
| | yeast count | |
| C. | Salmonella and Escherichia coli | Absence |
| 3. | Acidity or alkalinity | □ 0.05 ml of 0.1 N HCL |
| 4. | Loss of drying | □ 6.0% |
| 5. | Specific surface area | 0.05˜0.15 |
| 6. | Limit of chloride | □ 0.1% |
| 7. | Limit of sulfate | □ 1.0% |
| 8. | Lead | □ 0.001% |
| 9. | Relative content of stearic | Meet the |
| | Acid and palmitic acid | requirements of USP24 |
| 10. | Assay | 4.0˜5.0% Mg (dried basis) |

2.3 Technical specifications of starch

| | Items | Specifications |
|---|---|---|
| 1. | Identification | |
| A. | Solubility | A translucent, whitish jelly |
| B. | Color test | Reddish violet to deep blue |
| 2. | Microbial limits | |
| | Salmonella species and | Absence |
| | Escherichia coli | |
| 3. | pH | 4.5˜7.0 for Corn starch, |
| | | Tapioca starch and wheat |
| | | Starch; |
| | | 5.0˜8.0 for Potato starch |
| 4. | Loss of drying | □14.0% |
| 5. | Residue on ignition | □ 0.5% |
| 6. | Iron | □ 0.002% |
| 7. | Oxidizing substances | □ 0.002% |
| 8. | Sulfur dioxide | □ 0.008% |

2.4 Technical specification of Carboxymethylstach Sodium

| Items | Specifications |
|---|---|
| 1. Identification | |
| A. Color test | Add iodine indicator, produce blue |
| B. Sodium sact test | Conform |
| Acidity and alkalinity | 5.5˜7.5 |
| Total chlorine content | □ 3.5% (dried basis) |
| Loss of drying | □ 10.0% |
| Iron | □ 0.004% |
| Heavy metals | □ 0.002% |
| Assay | Contain Sodium 2.0˜4.0% |
| | (Calculated on dried basis) |

The daily dose of the composition of the present invention can vary over broad limits depending on several factors, e.g. on the activity of the active ingredients, the patient's condition and age, the severity of the disease.

The oral dose as a rule: usual dose; single dose is 200-250 mg; severe symptom; single dose is 400-500 mg, twice a day taken with boiled water. It has to be stressed that these doses figures are intended for information only, and administered dose must be determined each time by the physician therapeutist.

When healthy adults take orally 200 mg, 1.5-2 hours later the peak concentration will reach 1.21+−0.03 ug/ml; if take orally, 1.5-2 hours later the peak concentration will reach 2.73+−0.43 ug/ml. The half-life is four hours (t_{1/2}=4 h). In the majority of indications twice dosage may be taken orally. The product distribute mainly in bile, mucus, saliva, bone and prostate gland but the concentration is lower in brain tissue. It may be metabolized partly in liver and pharmaceutical concentration in urine may be retainable.

According to the further aspect of the present invention there is provided a process appropriate for preparing the compositions of the invention comprising the following steps:
i. Mixing the appropriate prescription amount of Ciprofloxacin Hydrochloride, Starch and Carboxymethyl starch into a container;
ii. Adding some amount of starch thick liquid and stir until a soft material formed in the previous step and then dry it at a temperature preferably 70 C for 4 hours;
iii. Granulating the soft material formed in the previous step and then dry it at a temperature preferably 70 C for 4 hours;
iv. Take it out and arrange the grain;
v. Adding some amount of Magnesium Stearate in order to fill well in a capsule using an automatic filling machine;

According to a preferred feature, the capsules are packaged in a blister foil. It does obtained compositions, which have excellent properties with regard to physical and microbial stability, without the need to use preservatives and are particularly appropriate for oral administration.

The following example is given for a better understanding of this description, without being deemed to be a limitation of the scope of the present invention.

### EXAMPLE I

Weight 200 kg of Ciprofloxacin Hydrochloride, 37.73 kg of starch and 12.00 kg of Carboxymethyl starch sodium into a container, mix well. Using 5.60 kg of starch and 80 kg of water prepare starch thick liquid (7%). Then add these liquids to the container until soft material is obtained. The total quantity of starch is 43.33 kg. After drying and arranging grains add 2.76 kg of Magnesium Stearate in order to fill well.

Through drying about 80 kg of water may be lost, but no ingredients may be removed and overage. Before filling capsules, the tester must sample and test. According to the test result adjust quantity of each capsule.

The technical specification of the present composition is shown by the following:

| Items | Specifications |
|---|---|
| Identification | The retention time of sample |
| A. The retention time test | corresponds to that of the standard. |
| B. TLC test | The result obtained from test |
| | solution corresponds to that of |
| | Standard Solution |
| Dissolution | Not less than 80% Uniformity |
| of dosage units | Meet the requirements |
| Assay | 90.0˜110.0% |

Details of the assay and other test procedure for finished product including data analysis:
[Identification]

A: The retention time of the major peak in the chromatogram of the Assay preparation corresponds to that of the standard preparation obtained as directed in the Assay.

B: Place a number of capsules, equivalent to about 1500 mg of Ciprofloxacin, in a suitable flask containing about 750 mL of water, and sonicate for about 20 minutes. Dilute with water to 1000 mL, add mix. Centrifuge a portion of this suspension, and use the clear supernatant solution obtained as the test solution. Dissolve a quantity of USP Ciprofloxacin Hydrochloride RS in water to obtain a standard solution containing 1.5 mg per ML. Proceed as directed for Identification test B under Ciprofloxacin Hydrochloride. Starting with Separately apply, as 1-cm bands, 5 uL each. “Except to use 10 uL each of the test solution and the standard solution: the specified result is obtained.

[Dissolution]

| Medium: | Water 900 | mL |
|---|---|---|
| Apparatus: | 50 | rpm |
| Time: | 30 | minutes |

Procedure—Determine the amount of Ciprofloxacin Hydrochloride (C₁₇H₁₈FN₃O₃.HCl) dissolved from ultraviolet absorbance at the wavelength of absorbance at about 276 nm of filtered portions of the solution under test, suitably diluted with Dissolution Medium, if necessary, in comparison with a standard solution having a known concentration of USP Ciprofloxacin Hydrochloride RS in the same medium.

Tolerances—An amount Of C₁₇H₁₈FN₃O₃. HCl equivalent to not less than 80% (Q) of the labeled amount of Ciprofloxacin C₁₇H₁₈FN₃O₃ is dissolved in 30 minutes.

Calculated Formula:

$$Q = \frac{{A}_{T} \times 900}{{A}_{S} \times L \times {D}_{S}} \times 100 \%$$

| | |
|---|---|
| A_{T} | Absorbance obtained from test Solution |
| A_{S} | Absorbance obtained from Standard Solution |
| D_{S} | Diluted multiple of Standard solution |
| L | Labeled quantity |

Uniformity of Dosage Units:

Take 20 pills Ciprofloxacin Hydrochloride capsules, weigh their internal drug, and the weight discrepancy is ±7.5%.

[Assay]

Mobile phase, Resolution solution and Chromatographic system—prepare as directed in the Assay under Ciprofloxacin Hydrochloride.

Standard preparation—Dissolve an accurately weighed quantity of USP Ciprofloxacin Hydrochloride RS quantitatively in water to obtain a solution having a known concentration of about 0.3 mg per mL.

Assay preparation—Transfer 5 capsules to a 500-mL volumetric flask, add about 400 mL of water, and sonicate for about 20 minutes. Dilute with water to volume, and mix. Dilute an accurately measured volume of this solution quantitatively with water to obtain containing the equivalent of about 0.25 mg of Ciprofloxacin per mL.

Procedure—proceed as directed for Procedure in the Assay under Ciprofloxacin Hydrochloride. Calculate the quantity in mg of Ciprofloxacin (C₁₇H₁₈FN₃O₃) in each capsule taken by the formula:
(331.35/367.81)(CL/D)(rL/rs)

In which 331.35 and 3 7.81 are the molecular weights of Ciprofloxacin and anhydrous Ciprofloxacin Hydrochloride, respectively, C is the concentration, in mg per mL, of USP Ciprofloxacin Hydrochloride RS in the Standard preparation, calculated on the anhydrous basis. L is the labeled quantity, in mg per mL of Ciprofloxacin in the Assay preparation, based on the labeled quantity per capsule and the extend of dilution, and rL and rs are the ciprofloxacin peak responses obtained from the Assay preparation and the Standard preparation, respectively.

Detailed report of stability studies to justify shell life (accelerated or long terms).

1. Long Term Testing

1.1 Scope

Three batches (980 01, 980 02, 980 03) of Ciprofloxacin Hydrochloride capsules have been subjected to stability tests under 20° C.±2° C. and 0% RH±5%. So far, two year' stability results are available.

1.2 Packaging

The container to be used is the same as the actual packaging used for storage and distribution.

1.3 Storage Condition

Temperature and humidity is 25° C.±2° C. and 0% RH±5%.

2. Accelerated Testing

2.1 Scope

Our factory has performed a new stability program according to the “Stability testing guideline for medicinal products in European Union”.

2.2 Packaging

The container to be used in the same as the actual packaging used for storage and distribution.

2.3 Storage Condition

Under 40° C.±2° C. and 75% RH±5% for 6 months.

3. Analytical Items

The following items were carried out in order to determine any changes in this product.

3.1. Appearance.

3.2. Dissolution.

3.3. Uniformity of dosage units.

3.4. Assay.

Item 3.1 was tested by estimation, items 3.2, 3.3, 3.4 were tested in accordance with section 7 “Test procedure for finished products”.

4. Results

Please see table I, II, III, IV.

5. Conclusion

In well-closed containers, no significant change was observed up on storage at 25° C.±2° C. and 60% RH±5% for two years. In no case degradation products were observed. We will continue long term testing in order to confirm the final validity of this product, but it should be up to two years.

TABLE I
Long term testing results of Ciprofloxacin
Hydrochloride capsules Batch No.: 980601

| Items | | Uniformity of | | |
|---|---|---|---|---|
| Month | Appearanced | dosage units | Dissolution | Assay |
| 0 | Off-white | Conform | 94% | 98.4% |
| | particle | | | |
| 3 | No change | Conform | 93.5% | 98.0% |
| 6 | No change | Conform | 93.2% | 98.1% |
| 9 | No change | Conform | 93.0% | 97.6% |
| 12 | No change | Conform | 93.3% | 97.5% |
| 18 | No change | Conform | 92.5% | 96.8% |
| 24 | No change | Conform | 92.1% | 97.0% |

TABLE II
Long term testing results of Ciprofloxacin
Hydrochloride capsules Batch No.: 980602

| Items | | Uniformity of | | |
|---|---|---|---|---|
| Month | Appearanced | dosage units | Dissolution | Assay |
| 0 | Off-white | Conform | 94.2% | 97.5% |
| | particle | | | |
| 3 | No change | Conform | 94.0% | 97.2% |
| 6 | No change | Conform | 94.1% | 97.3% |
| 9 | No change | Conform | 93.8% | 97.0% |
| 12 | No change | Conform | 93.6% | 96.9% |
| 18 | No change | Conform | 93.3% | 96.5% |
| 24 | No change | Conform | 93.2% | 96.6% |

TABLE III
Long term testing results of Ciprofloxacin
Hydrochloride capsules Batch No.: 980603

| Items | | Uniformity of | | |
|---|---|---|---|---|
| Month | Appearanced | dosage units | Dissolution | Assay |
| 0 | Off-white | Conform | 93.8% | 98.0% |
| | particle | | | |
| 3 | No change | Conform | 93.5% | 97.8% |
| 6 | No change | Conform | 93.6% | 97.6% |
| 9 | No change | Conform | 93.2% | 97.1% |
| 12 | No change | Conform | 93.4% | 97.0% |
| 18 | No change | Conform | 92.9% | 96.5% |
| 24 | No change | Conform | 92.6% | 96.3% |

TABLE IV
Accelerated testing results of Ciprofloxacin Hydrochloride
capsules Batch No.: 980601; 980602; 980603

| | | | Uniformity of | | |
|---|---|---|---|---|---|
| Storage Time | Batch No. | Apperance | Dosage units | Dissolution | Assay |
| Initial | 980601 | Conform | Conform | 94.0% | 98.4% |
| (15/06,1998) | 980602 | Conform | Conform | 94.2% | 97.5% |
| | 980603 | Conform | Conform | 93.8% | 98.0% |
| | 980601 | No change | Conform | 94.1% | 97.9% |
| I month | 980602 | No change | Conform | 94.0% | 97.2% |
| | 980603 | No change | Conform | 93.6% | 97.5% |
| | 980601 | No change | Conform | 93.7% | 97.4% |
| 2 month | 980602 | No change | Conform | 93.9% | 96.8% |
| | 980603 | No change | Conform | 93.6% | 96.9% |
| | 980601 | No change | Conform | 93.5% | 96.6% |
| 3 month | 980602 | No change | Conform | 93.4% | 96.1% |
| | 980603 | No change | Conform | 93.5% | 96.2% |
| | 980601 | No change | Conform | 93.0% | 96.0% |
| 6 month | 980602 | No change | Conform | 92.7% | 95.4% |
| | 980603 | No change | Conform | 92.8% | 95.0% |

Appearance of capsules: Off-white particle in hard capsule.

1. A pharmaceutical composition comprising the following essential components:
i. Ciprofloxacin Hydrochloride;
ii. Magnesium stearate;
iii. Starch; and
iv. Carboxymethylstach Sodium

2. The pharmaceutical composition of claim 1, the Ciprofloxacin Hydrochloride being in the amount of about 300 g of the composition.

3. The pharmaceutical composition of claim 1, the Magnesium stearate being in the amount of about 4 g of the composition.

4. The pharmaceutical composition of claim 1, the starch being in the amount of about 65 g of the composition.

5. The pharmaceutical composition of claim 1, the Carboxymethylstach being in the amount of 18 g of the composition.

6. A process for the preparation of a pharmaceutical composition containing Ciprofloxacin Hydrochloride comprising the following steps:
i. Mixing the appropriate prescription amount of Ciprofloxacin Hydrochloride, Starch and Carboxymethylstach into a container;
ii. Adding some amount of starch thick liquid and stir until a soft material is obtained;
iii. Granulating the soft material formed in the previous step and then dry at a temperature of 70° C. for 4 hours;
iv. Take it out and arrange the grain;
v. Adding some amount of Magnesium Stearate in order to fill well.

7. Use of the pharmaceutical composition according to claim 1 for the treatment of upper respiratory tract infections including tonsillitis, sinusitis, otitis media and pharynx inflammation; lower respiratory tract infections including acute and chronic bronchitis, bronchiectasis and pneumonia; urinary tract infections including urethritis, cystitis, pyelonephritis, prostates and pelvic inflammatory; gastrointestinal infections including enteric fever and infective diarrhea; skin and soft tissue infections; wounds infections; and infections caused by other sensitive bacteria.
